(19) United States
(12) Patent Application Publication
(10) Pub. No.: US 2020/0381080 A1
(43) Pub. Date: Dec. 3, 2020

(54) DISCOVERY OF ENGINEERED SEQUENCES

(71) Applicant: NOBLIS, INC., Reston, VA (US)

(72) Inventor: Sterling THOMAS, Woodbridge, VA (US)

(73) Assignee: NOBLIS, INC., Reston, VA (US)

(21) Appl. No.: 16/887,850

(22) Filed: May 29, 2020

Related U.S. Application Data
(60) Provisional application No. 62/855,749, filed on May 31, 2019.

(51) Int. Cl. G16B 20/20; C12Q 1/6869; G16B 20/30; G16B 50/30; G16B 25/10; G06N 20/20

(52) U.S. Cl. CPC G16B 20/20; C12Q 1/6869; G06N 20/20; G16B 50/30; G16B 25/10; G16B 20/30

(57) ABSTRACT

Techniques for determining whether a nucleic acid sequence is genetically engineered are provided. In some embodiments, a ratio is calculated based on a number of input reads that align with reference data, and the ratio is inputted into a classifier to determine whether the input reads represent an engineered sequence. In some embodiments, first output data is generated based on unassembled read-based comparison of nucleic acid data, while second output data is generated based on assembly-based comparison of the nucleic acid data; the first and second output data are inputted into a classifier to determine whether the input data represents an engineered sequence. In some embodiments, nucleic acid data is compared to three reference datasets representing (i) engineered sequences, (ii) evolutionary variations of an organism, and (iii) evolutionary variations of other organisms; a determination as to whether the input data represents an engineered sequence is based on the three comparisons.

### CROSS-REFERENCE TO RELATED APPLICATIONS

This application claims the benefit of U.S. Provisional Application No. 62/855,749, filed on May 31, 2019, the entire contents of which are incorporated herein by reference and for all purposes.

### STATEMENT REGARDING FEDERALLY-SPONSORED RESEARCH

This invention was made with government support under Contract No. N6600118C4506 awarded by Space and Naval Warfare Systems Command (SPAWAR). The government has certain rights in the invention.

### FIELD

The present disclosure relates generally to discovery of engineered sequences, and more specifically to determining whether an unknown organism has been engineered using an ensemble of solutions including machine-learning algorithms.

### BACKGROUND

There is a need for systems and methods for distinguishing between artificially engineered organisms (e.g., genetically modified organisms (GMOs)) and naturally occurring organisms. Such systems and methods are useful, for example, for detecting and quantifying genetically modified material within the food supply chain and have special relevance to traceability and labeling of the end product.

### SUMMARY

An exemplary computer-enabled method for determining whether an organism is genetically engineered comprises: receiving nucleic acid sequence data representing a plurality of input reads; comparing the nucleic acid sequence data for each read of the plurality of reads with a first set of nucleic acid sequence reference data, the first set of reference data representing a plurality of evolutionary variations of an organism; calculating, for each evolutionary variation of the plurality of variations of the organism, a ratio between a first number of the plurality of input reads over a second number of the plurality of input reads, wherein the first number is indicative of a number of the plurality of input reads that, based on the comparison, align with the first set of nucleic acid sequence reference data within a predefined range, and wherein the second number indicates a total number of the plurality of input reads; inputting the ratio into a machine-learning-based classifier to obtain data indicative of whether the plurality of input reads corresponds to one or more engineered organisms.

In some embodiments, the method further comprises obtaining the first number, wherein obtaining the first number comprises: comparing an input read of the plurality of input reads with nucleic acid sequence reference data corresponding to a respective evolutionary variation of the plurality of variations to obtain a degree of matching between the input read and the nucleic acid sequence reference data corresponding to the respective evolutionary variation; determining whether the degree of matching is within the predefined range; in accordance with a determination that the degree of matching is within the predefined range, incrementing the first number; and in accordance with a determination that the degree of matching is not within the predefined range, foregoing incrementing the first number.

In some embodiments, the predefined range comprises an upper threshold, and wherein the upper threshold is equal to or higher than 99%.

In some embodiments, the predefined range comprises a lower threshold, and wherein the lower threshold is equal to or lower than 95%.

In some embodiments, the data indicative of whether the plurality of input reads corresponds to one or more engineered organisms comprises one or more confidence scores.

An exemplary computer-enabled method for determining whether an organism is genetically engineered comprises: receiving nucleic acid sequence data representing a plurality of input reads for an organism; applying the nucleic acid sequence data for each read of the plurality of reads to a data structure representing an original pan-genome graph to generate a new pan-genome graph, wherein the data structure representing the pan-genome graph represents a plurality of variations of the organism; generating a feature vector based on a comparison of the new pan-genome graph to the original pan-genome graph; inputting the feature vector into a machine-learning-based classifier to obtain data indicative of whether the plurality of input reads corresponds to one or more engineered organisms.

In some embodiments, the feature vector comprises a plurality of components comprising information representing one or more features of the new pan-genome graph that differ from the original pan-genome graph.

In some embodiments, the one or more new features comprise one or more features selected from: a new cluster and a new edge.

In some embodiments, the one or more new features are based on a number of genes with unique alleles in clusters with penetrance of one or more predefined ranges.

In some embodiments, the one or more new features are based on a number of unique edges in the new pan-genome graph.

In some embodiments, the one or more new features are based on a number of clusters with penetrance of a predefined range.

An exemplary computer-enabled method for determining whether an organism is genetically engineered comprises: receiving nucleic acid sequence data representing a plurality of input reads for an organism; performing an unassembled read-based comparison of the nucleic acid sequence data against a first set of nucleic acid sequence reference data to generate first output data; performing an assembly-based comparison of the nucleic acid sequence data against a second set of nucleic acid sequence reference data to generate second output data; inputting the first output data and the second output data into one or more machine-learning-based classifiers to obtain data indicative of whether the plurality of input reads corresponds to one or more engineered organisms.

In some embodiments, the first set of nucleic acid sequence reference data represents a plurality of evolutionary variations of the organism.

In some embodiments, performing an unassembled read-based comparison of the nucleic acid sequence data against a first set of nucleic acid sequence reference data to generate first output data comprises: calculating, for each evolutionary variation of the plurality of variations of the organism, a ratio between a first number of the plurality of input reads over a second number of the plurality of input reads, wherein the first number is indicative of a number of the plurality of input reads that, based on the comparison, align with the first set of nucleic acid sequence reference data within a predefined range, and wherein the second number indicates a total number of the plurality of input reads.

In some embodiments, performing an assembly-based comparison of the nucleic acid sequence data against a second set of nucleic acid sequence reference data to generate second output data comprises: constructing a pan-genome graph based on the nucleic acid sequence data.

In some embodiments, the method further comprises inputting the first output data into a first machine-learning-based classifier to obtain a first decision value and a first confidence score; inputting the second output data into a second machine-learning-based classifier to obtain a second decision value and a second confidence score; wherein the data indicative of whether the plurality of input reads corresponds to one or more engineered organisms is based on the first decision value, the second decision value, the first confidence score, and the second confidence score.

An exemplary computer-enabled method for determining whether an organism is genetically engineered comprises: receiving nucleic acid sequence data representing a plurality of input reads; comparing the nucleic acid sequence data to a first set of nucleic acid sequence reference data, the first set of reference data representing a plurality of reference nucleic acid sequences associated with genetic engineering; comparing the nucleic acid sequence data to a second set of nucleic acid sequence reference data, the second set of reference data representing a plurality of reference nucleic acid sequences associated with a plurality of evolutionary variations of a first organism; comparing the nucleic acid sequence data to a third set of nucleic acid sequence reference data, the third set of reference data representing a plurality of reference nucleic acid sequences associated with a plurality of organisms different from the first organism; based on one or more of the comparisons, generating and storing data indicating whether the plurality of input reads corresponds to one or more engineered organisms.

In some embodiments, the method further comprises determining a degree of matching between the nucleic acid sequence data and the first set of nucleic acid sequence reference data; in accordance with a determination that the degree of matching is below a threshold, comparing the nucleic acid sequence data to the second set of nucleic acid sequence reference data, and in accordance with a determination that the degree of matching is not below a threshold, foregoing comparing the nucleic acid sequence data to the second set of nucleic acid sequence reference data.

An exemplary non-transitory computer-readable storage medium stores one or more programs, the one or more programs comprising instructions, which when executed by one or more processors of an electronic device having a display, cause the electronic device to: receiving nucleic acid sequence data representing a plurality of input reads; comparing the nucleic acid sequence data for each read of the plurality of reads with a first set of nucleic acid sequence reference data, the first set of reference data representing a plurality of evolutionary variations of an organism; calculating, for each evolutionary variation of the plurality of variations of the organism, a ratio between a first number of the plurality of input reads over a second number of the plurality of input reads, wherein the first number is indicative of a number of the plurality of input reads that, based on the comparison, align with the first set of nucleic acid sequence reference data within a predefined range, and wherein the second number indicates a total number of the plurality of input reads; inputting the ratio into a machine-learning-based classifier to obtain data indicative of whether the plurality of input reads corresponds to one or more engineered organisms.

An exemplary non-transitory computer-readable storage medium stores one or more programs, the one or more programs comprising instructions, which when executed by one or more processors of an electronic device having a display, cause the electronic device to: receiving nucleic acid sequence data representing a plurality of input reads for an organism; applying the nucleic acid sequence data for each read of the plurality of reads to a data structure representing an original pan-genome graph to generate a new pan-genome graph, wherein the data structure representing the pan-genome graph represents a plurality of variations of the organism; generating a feature vector based on a comparison of the new pan-genome graph to the original pan-genome graph; inputting the feature vector into a machine-learning-based classifier to obtain data indicative of whether the plurality of input reads corresponds to one or more engineered organisms.

An exemplary non-transitory computer-readable storage medium stores one or more programs, the one or more programs comprising instructions, which when executed by one or more processors of an electronic device having a display, cause the electronic device to: receiving nucleic acid sequence data representing a plurality of input reads for an organism; performing an unassembled read-based comparison of the nucleic acid sequence data against a first set of nucleic acid sequence reference data to generate first output data; performing an assembly-based comparison of the nucleic acid sequence data against a second set of nucleic acid sequence reference data to generate second output data; inputting the first output data and the second output data into one or more machine-learning-based classifiers to obtain data indicative of whether the plurality of input reads corresponds to one or more engineered organisms.

An exemplary non-transitory computer-readable storage medium stores one or more programs, the one or more programs comprising instructions, which when executed by one or more processors of an electronic device having a display, cause the electronic device to: receiving nucleic acid sequence data representing a plurality of input reads; comparing the nucleic acid sequence data to a first set of nucleic acid sequence reference data, the first set of reference data representing a plurality of reference nucleic acid sequences associated with genetic engineering; comparing the nucleic acid sequence data to a second set of nucleic acid sequence reference data, the second set of reference data representing a plurality of reference nucleic acid sequences associated with a plurality of evolutionary variations of a first organism; comparing the nucleic acid sequence data to a third set of nucleic acid sequence reference data, the third set of reference data representing a plurality of reference nucleic acid sequences associated with a plurality of organisms different from the first organism; based on one or more of the comparisons, generating and storing data indicating whether the plurality of input reads corresponds to one or more engineered organisms.

An electronic device comprises: one or more processors; a memory; and one or more programs, wherein the one or more programs are stored in the memory and configured to be executed by the one or more processors, the one or more programs including instructions for: receiving nucleic acid sequence data representing a plurality of input reads; comparing the nucleic acid sequence data for each read of the plurality of reads with a first set of nucleic acid sequence reference data, the first set of reference data representing a plurality of evolutionary variations of an organism; calculating, for each evolutionary variation of the plurality of variations of the organism, a ratio between a first number of the plurality of input reads over a second number of the plurality of input reads, wherein the first number is indicative of a number of the plurality of input reads that, based on the comparison, align with the first set of nucleic acid sequence reference data within a predefined range, and wherein the second number indicates a total number of the plurality of input reads; inputting the ratio into a machine-learning-based classifier to obtain data indicative of whether the plurality of input reads corresponds to one or more engineered organisms.

An electronic device comprises: one or more processors; a memory; and one or more programs, wherein the one or more programs are stored in the memory and configured to be executed by the one or more processors, the one or more programs including instructions for: receiving nucleic acid sequence data representing a plurality of input reads for an organism; applying the nucleic acid sequence data for each read of the plurality of reads to a data structure representing an original pan-genome graph to generate a new pan-genome graph, wherein the data structure representing the pan-genome graph represents a plurality of variations of the organism; generating a feature vector based on a comparison of the new pan-genome graph to the original pan-genome graph; inputting the feature vector into a machine-learning-based classifier to obtain data indicative of whether the plurality of input reads corresponds to one or more engineered organisms.

An electronic device comprises: one or more processors; a memory; and one or more programs, wherein the one or more programs are stored in the memory and configured to be executed by the one or more processors, the one or more programs including instructions for: receiving nucleic acid sequence data representing a plurality of input reads for an organism; performing an unassembled read-based comparison of the nucleic acid sequence data against a first set of nucleic acid sequence reference data to generate first output data; performing an assembly-based comparison of the nucleic acid sequence data against a second set of nucleic acid sequence reference data to generate second output data; inputting the first output data and the second output data into one or more machine-learning-based classifiers to obtain data indicative of whether the plurality of input reads corresponds to one or more engineered organisms.

An electronic device comprises: one or more processors; a memory; and one or more programs, wherein the one or more programs are stored in the memory and configured to be executed by the one or more processors, the one or more programs including instructions for: receiving nucleic acid sequence data representing a plurality of input reads; comparing the nucleic acid sequence data to a first set of nucleic acid sequence reference data, the first set of reference data representing a plurality of reference nucleic acid sequences associated with genetic engineering; comparing the nucleic acid sequence data to a second set of nucleic acid sequence reference data, the second set of reference data representing a plurality of reference nucleic acid sequences associated with a plurality of evolutionary variations of a first organism; comparing the nucleic acid sequence data to a third set of nucleic acid sequence reference data, the third set of reference data representing a plurality of reference nucleic acid sequences associated with a plurality of organisms different from the first organism; based on one or more of the comparisons, generating and storing data indicating whether the plurality of input reads corresponds to one or more engineered organisms.

### BRIEF DESCRIPTION OF THE FIGURES

The patent or application file contains at least one drawing executed in color. Copies of this patent or patent application publication with color drawings will be provided by the office upon request and payment of the necessary fee.

FIG. 1 depicts an exemplary platform for determining whether an unknown organism has been engineered, in accordance with some embodiments.

FIG. 2 depicts an exemplary process performed by the read-based analysis component, in accordance with some embodiments.

FIG. 3A depicts an exemplary list of software used for QC and genome assembly, in accordance with some embodiments.

FIG. 3B depicts exemplary assembly statistics for a plurality of samples, in accordance with some embodiments.

FIG. 4 depicts exemplary metagenomics output, in accordance with some embodiments.

FIG. 5 depicts exemplary metagenomics output, in accordance with some embodiments.

FIG. 6 depicts exemplary metagenomics output, in accordance with some embodiments.

FIG. 7 depicts exemplary metagenomics output, in accordance with some embodiments.

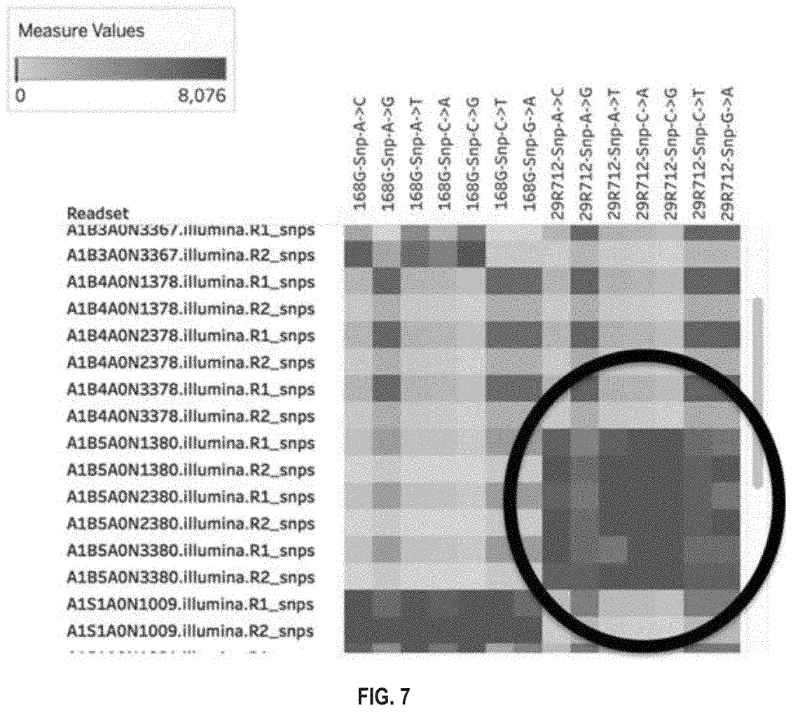

FIG. 8A depicts an exemplary process for determining whether an organism is engineered, in accordance with some embodiments.

FIG. 8B depicts an exemplary process for determining whether an organism is engineered, in accordance with some embodiments.

FIG. 8C depicts an exemplary process for determining whether an organism is engineered, in accordance with some embodiments.

FIG. 8D depicts an exemplary process for determining whether an organism is engineered, in accordance with some embodiments.

FIG. 9 depicts an exemplary electronic device in accordance with some embodiments.

### DETAILED DESCRIPTION

Disclosed herein are methods, electronic devices, user interfaces, systems, and non-transitory computer-readable storage media for determining whether an unknown organism has been engineered. In some embodiments, an exemplary platform comprises at least three components: a read-based analysis component (e.g., using the BioVelocity platform), an assembled-reads-based analysis component (e.g., whole genome alignment using the PanOCT platform), and a machine-learning-based classifier (e.g., a trained neural network).

In general, detection methods for GMOs fall into two classes: DNA-based and immunological-based. DNA-based methods include quantitative polymerase chain reaction (qPCR), multiplexed PCR, and targeted sequencing. Immunological detection methods include lateral flow assays and enzyme-linked immunosorbent assays (ELISA), typically made with monoclonal antibodies specific for the antigen of interest. Both classes of methodologies generally rely on a priori knowledge of the sequence mutation (PCR, targeted sequencing) or the expressed gene product (immunological detection). The most common transgenic elements found in GMOs, such as the p35S promoter (from cauliflower mosaic virus), have been used as signatures of engineering. In general, the preferred detection methodologies lately have been DNA-based, largely on a PCR platform (e.g., qPCR, multiplex PCR) due to the speed and ability to multiplex assays.

When faced with an unknown genomic modification, whole genome sequencing allows for full characterization of a sample without any prior knowledge of the mutation, as was demonstrated with a mutant rice strain. However, the success of this strategy is predicated upon the availability of good reference genomes for specific varieties of organisms. Recent developments in NGS technologies and capabilities, embraced and leveraged in our proposal, make the generation of whole genome sequences more straightforward, accurate, and rapid than previously possible, addressing this potential limitation. The DNA sequence-based approaches described herein do not depend on a priori knowledge of genome alterations. By using the PGG analysis and multiple reference sequence comparisons, we more effectively identify genetic region anomalies and potentially novel proteins that comprise the best candidates representing engineered sequences—all while leveraging BioVelocity's ability to perform complex, massively parallel sequence comparisons to identify genetic region anomalies efficiently and down-select the dataset to the most likely engineered sequences.

Our comprehensive approach uses innovative multi-omics platforms, including metabolomics, which goes beyond methods historically used for detection of GMOs. We propose a novel, antibody-free platform for detection of known proteins and characterization of unknown proteins.

Each biological and computational technology and analysis methodology can stand alone, although they interconnect to enhance and complement each other. Ultimately, all outputs are integrated into an analysis tool to predict if organisms in a dataset have been edited.

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

Although the following description uses terms “first,” “second,” etc. to describe various elements, these elements should not be limited by the terms. These terms are only used to distinguish one element from another. For example, a first graphical representation could be termed a second graphical representation, and, similarly, a second graphical representation could be termed a first graphical representation, without departing from the scope of the various described embodiments. The first graphical representation and the second graphical representation are both graphical representations, but they are not the same graphical representation.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

The term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

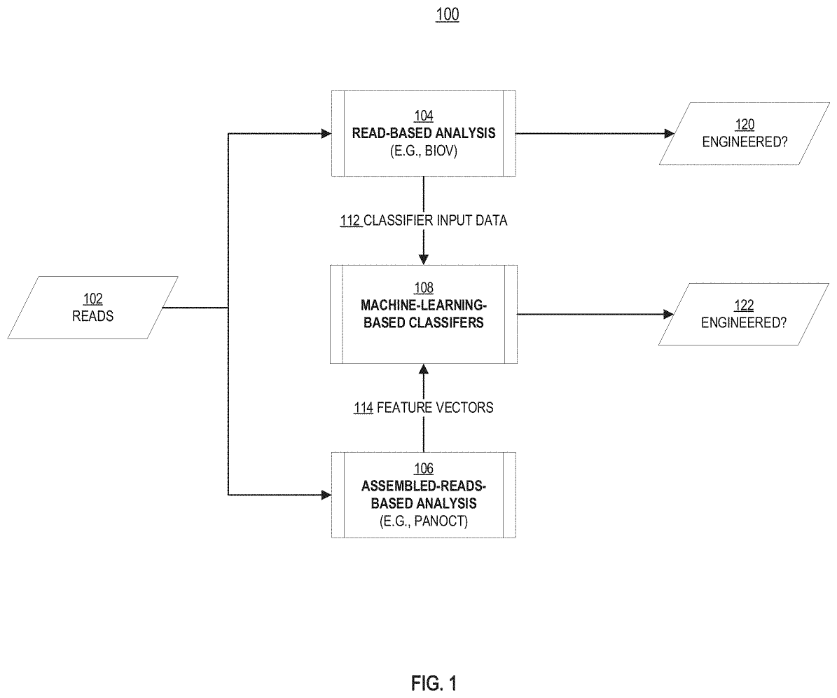

FIG. 1 depicts an exemplary platform 100 for determining whether an unknown organism has been engineered, in accordance with some embodiments. In some embodiments, platform 100 may comprise one or more software tools that may be implemented on one or more processors. The platform receives genomic data, and the platform may be configured to determine whether the genomic data received by the platform represents organisms that evolved naturally (e.g., are classified as naturally occurring evolutionary strains) or organisms were engineered by manual human intervention (e.g., are classified as genetically-engineered strains). In some embodiments, the genomic data received by the platform is in the form of a plurality of reads 102. In some embodiments, the plurality of reads 102 correspond to one or more paired-end samples containing one or more strains (e.g., the Bacillus subtilis subtilis strain). Each of the one or more strains may be a wild type (e.g., naturally evolutionarily occurring) strain or a product of genetic engineering. As described below, the platform 100 analyzes the raw reads 102 using a read-based analysis component 104 (e.g., BioV), an assembled-reads-based analysis component 106 (e.g., PanOCT), and a machine-learning-based classifier 108. The platform may be configured to generate and output (or store) data representing results 120 and 122 from the various components, and said results may indicate whether one or more strains in the received genomic data is an engineered or wild type strain. In some embodiments, the results are on the organism level. In some embodiments, the results on the sample level.

In some embodiments, the results 120 and 122 are reviewed and evaluated by one or more human experts. Additionally or alternatively, the results 120 and 122 are automatically aggregated by the platform to provide a determination (i.e., engineered or not) and a confidence level associated with the determination (e.g., said determination may be provided in the form of one or more stored, transmitted, and/or displayed indicators regarding a determined classification and/or confidence level).

With reference to FIG. 1, the platform 100 provides the plurality of reads 102 to the read-based analysis component 104 to obtain result 120. In some embodiments, the read-based analysis component 104 is a software tool configured to compare genomic read data to reference data, wherein the reference data may be provided in the form of one or more reference databases or reference indices. In some embodiments, the reference data may be data that represents nucleic acid sequences for one or more known organisms. In some embodiments, the reference data may be provided as one or more reference indices, wherein each of the one or more reference indices may comprise a plurality of elements (e.g., entries in the index) corresponding to a respective permutations of nucleic acid sequences. In some embodiments, each of the elements may represent a permutation of a predefined base length (e.g., 16). In some embodiments, an index may comprise reference numbers indicating position data in a genomic sequence corresponding to a specific element of the index (e.g., indicating that that the permutation shown by that element appears at the indicated position in a certain genome). In some embodiments, an index may be configured to represent a single genome (e.g., by including reference numbers indicating position data only for that single genome) or may be configured to represent a plurality of genomes (e.g., by including reference numbers indicating position data for a plurality of genomes). In some embodiments, read data (e.g., data input to platform 100) may be compared to the data represented in the one or more indexes of component 104 to determine whether one or more of the reads does not align, partially aligns, or perfectly aligns with the genomic reference data represented by the one or more indexes. In some embodiments, the read-based analysis component 104 comprises the BioVelocity platform (“BioV”), including one or more reference indices provided thereby. Additional descriptions of the BioV platform are provided in U.S. Pat. No. 10,191,929 and U.S. application Ser. No. 14/718,950, the content of which are incorporated by reference in their entirety.

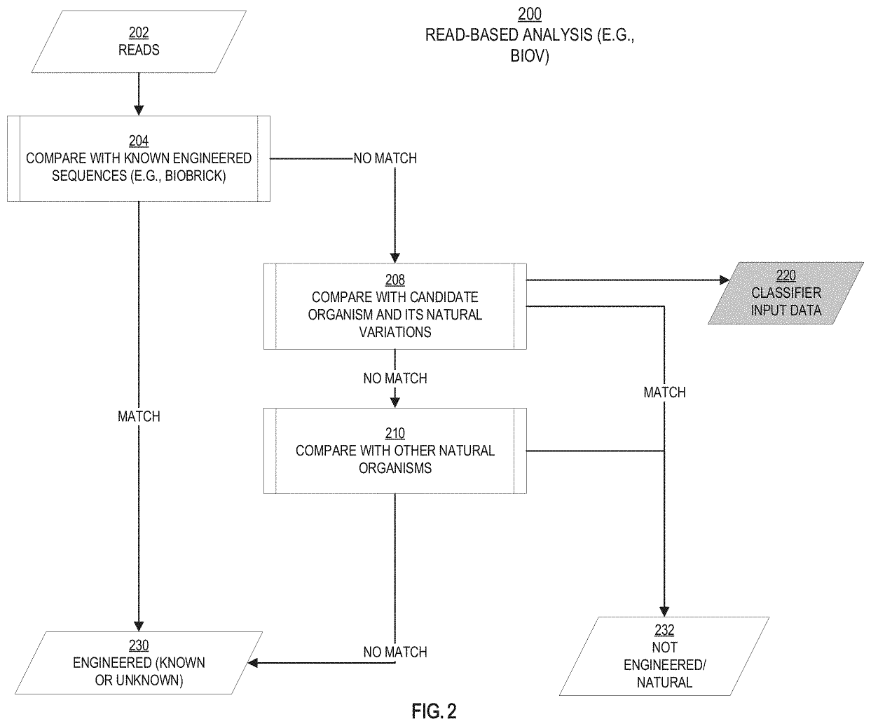

FIG. 2 provides an exemplary process 200 performed by the read-based analysis component 104, in accordance with some embodiments. Process 200 is performed, for example, using one or more electronic devices implementing a software platform. In some examples, process 200 is performed using a client-server system, and the blocks of process 200 are divided up in any manner between the server and a client device. In some examples, the blocks of process 200 are divided up between the server and multiple client devices. In other examples, process 200 is performed using only a client device or only multiple client devices. In process 200, some blocks are, optionally, combined, the order of some blocks is, optionally, changed, and some blocks are, optionally, omitted. In some examples, additional steps may be performed in combination with the process 200. Accordingly, the operations as illustrated (and described in greater detail below) are exemplary by nature and, as such, should not be viewed as limiting.

With reference to FIG. 2, a system (e.g., one or more electronic devices) receives a plurality of reads 202 representing genomic data of one or more organisms. The plurality of reads 202 can be the same or a subset of the plurality of reads 102 of FIG. 1. In some embodiments, multiple copies (e.g., 30-50) of an entire genome are represented in the plurality of reads 202, which are overlapping and interlinking with each other. In some embodiments, the system performs the process 200 without assembling any of the plurality of reads 202.

As discussed below, the system compares the plurality of reads 202 with a plurality of sets of genomic reference data at blocks 204, 206, and 208. Herein, the sets of genomic reference data are referred to as indices (e.g., BioV indices); however, in some embodiments, one or more of the sets of genomic reference data may take another form. As discussed above, the indices comprise data representing various repositories or collections of reference nucleic acid sequences of one or more organisms. The indices can be pre-built or constructed on-the-fly based on characteristics of the plurality of reads.

In some embodiments, before block 204, the system compares the reads with known markers of engineering, such as antimicrobial resistance markers and radical toxins (without horizontal transfer). If the match between the reads and the known markers is above a predefined threshold, the system determines that the organism is a product of genetic engineering. If the match does not exceed the predefined threshold, the system proceeds to block 204 to further analyze the plurality of reads 202.

At block 204, the system compares each of the one or more of the plurality of reads 202 against a first set of reference genomic data, for example by comparing the one or more reads against a first index. In some embodiments, the first set of reference genomic data comprises one or more nucleic acid sequences known to have been engineered, known to be used in genetic engineering of certain organisms, and/or otherwise known to be indicative of genetic engineering. The first set of reference genomic data may comprise are collections of genetically engineered components that have been published or voluntarily deposited. In some embodiments, the first set of reference genomic data may comprise genomic data from BioBrick, iGEM, or a combination thereof. For example, the first set of reference genomic data may comprise a collection of all published BioBrick sequences, excluding those labeled as naturally occurring in one or more selected organisms (e.g., Bacillus subtilis). In some embodiments, naturally occurring nucleic acid sequences from one organism that are known to be used in genetic engineering of nucleic acid sequences of other organisms may be included in the first set of reference genomic data.

The system determines whether each of the plurality of reads 202 matches first set of reference genomic data, for example by comparing the one or more reads to the first index. In some embodiments, the system determines whether the similarity between each read of the plurality of reads 202 and one or more nucleic acid sequences represented in the first index exceeds a predefined threshold (e.g., a percentage of alignment). In some embodiments, the predefined threshold is 95%. That is, the system allows mismatching of no more than 5% of the total length of the sequence. Accordingly, for each read of the plurality of reads 202, the platform determines whether it is considered a match with a reference sequence in the first index.

In some embodiments, the platform determines that, among the plurality of reads 202, there are m reads matching references sequences corresponding to an engineered organism, and n reads that do not. If the ratio between m and the total number of reads exceeds a predefined threshold, the platform determines that the sample is an engineered sample and proceeds to block 230. If the ratio between m and the total number of reads does not exceed the predefined threshold, the platform proceeds to block 208. In some embodiments, the predefined threshold is specific to the organism, the sequencing technology, or a combination thereof. In some embodiments, the determination is on a sample level. In some embodiments, the determination is on an organism level.

At block 208, the system compares some or all of the plurality of reads 202 with set of reference genomic data, for example by comparing the one or more reads to a second index. In some embodiments, the system compares each of the plurality of reads 202 with the second index. In some embodiments, the system analyzes only a subset of the plurality of reads 202 in block 208. For example, at block 208, the system analyzes only the reads that do not match the first index in block 204.

In some embodiments, the second set of reference genomic data comprises a collection of reference nucleic acid sequences relating to a candidate organism and, optionally, to natural variation(s) of the candidate organism that are known to be possible through organic evolution and/or are predetermined to be likely to be possible through organic evolution. In some embodiments, the candidate organism is identified based on a priori knowledge of the source of the plurality of reads 202. For example, if the system knows that the plurality of reads 202 corresponds to Bacillus subtilis, the system may use a set of reference genomic data (e.g., second index) at block 208 that represents various natural variants of Bacillus subtilis that are either known or determined to be possible through natural evolution of the organism. In one example, the second index comprises data representing 146 Bacillus subtilis subtilis reference strains.

At block 208, the system compares each read with the second set of reference genomic data, for example by comparing each read to the second index. In some embodiments, the system determines whether the similarity between each read and the reference strains represented in the second index exceeds a predefined threshold (e.g., a percentage of alignment). Accordingly, for each read of the plurality of reads 202, the platform determines whether it is considered a match with a reference sequence in the second index.

In some embodiments, the platform determines that, among the total number of reads being compared against the second index, there are m reads matching references sequences corresponding to an engineered organism. If the ratio between m and the total number of reads exceeds a predefined threshold, the platform determines that the sample is not an engineered sample and proceeds to block 232. If the ratio between m and the total number of reads does not exceed the predefined threshold, the platform proceeds to block 210. The predefined threshold may be different from the one used in block 204. In some embodiments, the predefined threshold is specific to the organism, the sequencing technology, or a combination thereof. In some embodiments, the determination is on a sample level. In some embodiments, the determination is on an organism level.

The comparison at block 208 produces a count of reads that are perfectly aligned, partially aligned, and not aligned to the second set of reference genomic data (e.g., the second index). In some embodiments, partially alignment is defined as an alignment that is equal to or greater than a lower threshold amount, while equal to or less than an upper threshold amount. In some embodiments, the lower threshold amount may be 50%, 75%, 80%, 90%, 95%, 98%, or 99%. In some embodiments, the upper threshold amount may be 95%, 98%, 99%, or any percentage alignment defined by alignment of all bases of the read except a non-zero number of bases, such as one base, two bases, or three bases. For a partially aligned read, an alignment score can be calculated based on the percentage of alignment between the read and the reference sequence. In some embodiments, the references that partially aligned are reported with alignment scores and single nucleotide polymorphisms (“SNPs”) are reported.

Further, a summary statics analysis can be run to calculate a ratio of the number of partially aligned reads to the number of reads included in the input genomic data (e.g., the total number of reads in reads 202). The ratio may be provided as input to a classifier in the platform (e.g., classifier input data 220 supplied to classifier 108) that may be used to determine whether an organism represented by the input genomic data is natural or engineered. With reference to FIGS. 1 and 2, the calculated ratios are provided as classifier input data 220 in FIG. 2 and classifier input data 112 in FIG. 1. In some embodiments, the input data 112 comprises multiple ratios corresponding to multiple variants in block 208. The ratio of partially aligned reads to the total number of reads inputted may be calculated for each one of the unique strains represented by the second set of reference genomic data; for example, if the second index represents 146 strains of Bacillus subtilis subtilis, then 146 ratios may be calculated, with each ratio based on the number of reads in the read data that partially aligned to a specific respective strain of Bacillus subtilis subtilis.

At block 210, the system compares some or all of the plurality of reads 202 with a third set of reference genomic data, for example by comparing the one or more reads to a third index. In some embodiments, the system compares each of the plurality of reads 202 with the second index. In some embodiments, the system analyzes only a subset of the plurality of reads 202 in block 210. For example, at block 210, the system analyzes only the reads that do not match the first index in block 204 and do not match the second index in block 208.

In some embodiments, the third set of reference genomic data comprises data representing nucleic acid sequences known to be associated with a plurality of known natural organisms, for example in addition to the single candidate organism represented in some embodiments by the second set of reference genomic data. In some embodiments, the third set of reference genomic data may be a metagenomic data set. In some embodiments, the third set of reference genomic data (e.g., third index) includes data representing a collection of NCBI's bacterial nucleic acid sequences using the GenBank collection of complete chromosomes and plasmids. In some embodiments, the comparison performed at block 210 may cast a wider net than the comparison performed at block 208, such that it may be computationally advantageous to perform the comparison at block 208 first, for example based on preexisting knowledge about the likelihood that the input reads are associated with a specific candidate organism represented by the reference data at block 208.

At block 210, the system compares each read with the third set of reference genomic data, for example by comparing each read to the third index. In some embodiments, the system determines whether the similarity between each read and the reference sequence represented in the third index exceeds a predefined threshold (e.g., a percentage of alignment). Accordingly, for each read, the platform determines whether it is considered a match with a reference sequence in the third index.

In some embodiments, the platform determines that, among the total number of reads being compared against the third index, there are m reads matching reference sequences corresponding to the third index. If the ratio between m and the total number of reads exceeds a predefined threshold, the platform determines that the sample is not an engineered sample and proceeds to block 232. If the ratio between m and the total number of reads does not exceed the predefined threshold, the platform proceeds to block 230. In some embodiments, the predefined threshold is specific to the organism, the sequencing technology, or a combination thereof. In some embodiments, the predefined threshold is different from the one used in block 204 and/or the one used in block 208. In some embodiments, the determination is on a sample level. In some embodiments, the determination is on an organism level.

In some embodiments of the process 200, block 208 is omitted. In some embodiments, the system allows a user to specify whether block 208 is to be omitted. In some embodiments, the system automatically determines whether to omit block 208, for example, based on the composition of the plurality of reads 202. For example, if the plurality of reads 202 are known to be or suspected to be largely from a single species (e.g., over 80%, over 90%), then block 208 may be included to increase computational efficiency as discussed above. In some embodiments, block 208 may be included for the purpose of generating classifier input data 220 as discussed above. In some embodiments, block 210 is performed before block 208. Specifically, the system can first perform block 210 to identity one or more candidate organisms, and then run the organism-specific comparison in block 208.

In some embodiments, analysis at block 208 may further comprise accounting for the possibility that reads 202 comprise a relatively small number of engineered reads and a relatively larger number of naturally-occurring reads; this kind of sample may be referred to as a diluted sample. In some embodiments, a diluted sample may cause a system to determine that reads that are actually engineered may only appear to be engineered due to their being outliers or being represented by bad data. Thus, to correct for this error, block 208 may, in some embodiments, comprise the additional step of comparing the input reads to the second set of genomic reference data using a lower threshold than the initial threshold used at block 208. That is, following comparison of the read data at block 208 using a first threshold, and following a determination that an insufficient number of the input reads exceed the first threshold for the system to determine that the input reads represents an engineered organism, the system may then compare the input reads to a second, lower threshold. In some embodiments, if an insufficient number of the input reads exceed the first threshold, but if a sufficient number of reads (e.g., a second predetermined threshold number of reads that must exceed the lower threshold, larger than a first predetermined number of reads that must exceed the higher threshold) exceed the lower threshold, then the system may nonetheless determine that the input reads represents an engineered organism.

Turning back to FIG. 1, the platform 100 comprises assembled-reads-based analysis component 106. The assembled-reads-based analysis component can assemble some of the plurality of reads 202 into one or more assemblies and compare the assemblies against reference genomic data for a known candidate organism. In some embodiment, unlike read-based analysis component 104, assembled-reads-based analysis component 106 requires prior knowledge of a specific candidate organism for comparison. In some embodiments, unlike read-based analysis component 104, assembled-reads-based analysis component 106 may not be used for metagenomic samples. Based on prior knowledge of the candidate organism, the system compares the assemblies against data representing nucleic acid sequences associated with the candidate organism. Where there is a deletion or an insertion or a rearrangement of genes, the component provides a numeric value indicative of the difference.

In some embodiments, the component 106 (e.g., PanOCT) performs whole genome alignment and build a network graph (i.e., a pan-genome graph) of how the whole genomes relate to each other. For example, if there are a first version of the Phila subtilis that has two additional genes and a second version of the Phila subtilis, the network graph would include a line between those two and represent those by two deletions from going from one to the other. In some embodiments, the component 106 conducts an exhaustive comparison and constructs one or more network graphs for a collection of reference nucleic acid sequences relating to a candidate organism and, optionally, to natural variation(s) of the candidate organism that are known to be possible through organic evolution and/or are predetermined to be likely to be possible through organic evolution. For example, for Bacillus subtilis, the component 106 may process data representing various natural variants of Bacillus subtilis that are either known or determined to be possible through natural evolution of the organism (e.g., 146 Bacillus subtilis subtilis reference strains). Based on the result graph, the system identifies medoids in the pan-genome graph.

The system compares the plurality of reads 102 against that pan-genome graph and identifies new clusters and new edges, which are indicative of new organisms. The system can then obtain the count of the new clusters and new edges.

The component 106 further generates feature vectors 114 to pass relevant information to the classifier 108 as training data or input data to recognize synthetic engineering versus natural occurrence. In some embodiments, the features include: the number of unique clusters as annotated by the graph, the number of genes with unique alleles in clusters with penetrance of a first predefined range (e.g., 0-25%), the number of genes with unique alleles in clusters with penetrance of a second predefined range (e.g., 25-75%), the number of genes with unique alleles in clusters with penetrance of a third predefined range (e.g., 75-100%), the number of unique edges, the number of edges with unique alleles in clusters with penetrance of a first range (e.g., 0-25%), the number of edges with unique alleles in clusters with penetrance of a second range (e.g., 25-75%), the number of edges with unique alleles in clusters with penetrance of a third range (e.g., 75-100%), the number of genes that are shorter than expected, the number of genes that are longer than expected, the number of edges that are shorter than expected, the number of edges that are longer than expected, the number of protein coding genes which appear to be frameshifted, clusters with penetrance of a predefined range (e.g., 75% or higher) which are missing (not including core), edges with penetrance of a predefined range (e.g., 75% or higher) which are missing (not including core), singleton core clusters which are missing, core edges which are missing, number of (nonsingleton) shared clusters found includes core, number of (nonsingleton) shared edges found includes core, number of genes whose ANI is less than 90% to the cluster medoid, number of rearrangements (novel core edges), ANI measured against all of the cluster medoids, minimum edit distance to nearest allele in the PGG, how many “column” differences have not been seen in the PGG, or any combination thereof.

With reference to FIG. 1, the platform 100 further comprises the machine-learning-based classifier 108. The classifier is an algorithm or model (e.g., a neural network) that is configured to receive input data from the read-based analysis component 104 (e.g., the ratios of partial alignments to the total number of reads) and the assembled-reads-based analysis component 106 (e.g., feature vectors) and provide an output indicating whether the organism is natural or engineered.

In some embodiments, the classifier comprises a plurality of classifiers, wherein a first subset of the classifiers is configured to receive input data from the component 104 while a second subset of the classifiers is configured to receive input data from the component 106. In some embodiments, each classifier is configured to output a binary decision (e.g., engineered or not engineered) and a confidence score associated with the binary decision. The outputs of the plurality of classifiers can be aggregated by the system to produce a single decision (e.g., a single indication of whether the system has determined that the input reads represent an organism that is most likely natural or most likely engineered) and an associated confidence score.

### Exemplary Realization

An exemplary platform has been implemented to determine if an unknown organism has been engineered. This platform includes read-based analysis using BioVelocity (BioV), whole genome alignment using PanOCT, and an artificial neural network classifier.

In some embodiments, the BioV process was the only step making independent outcome decisions in addition to feeding data to the classifier. This avoids over-weighting the BioV output, data that was fed to the classifier was excluded during the independent BioV outcome calling process. For example, the partially aligned reads are not used to make an independent call if they are then inputted to the classifier. NLP output could also be excluded if the classifier was trained for that data type.

### Exemplary Realization—Input Data

In one exemplary realization, 50 pair-end samples from IARPA containing the Bacillus subtilis subtilis strain were received. Some of the strains are wild type strains while others contained genetic engineering. The strains were sequenced by IARPA using both Illumina and MinION technologies and the raw read sequences were delivered. In total, 150 reads were delivered, including 100 reads from the Illumina sequencer and 50 from the MinION. In all, there were three read-sets per sample.

The raw reads were assembled and analyzed using both BioV and PanOCT. The results of the different analysis processes were used by the implemented platform to determine whether the strain was an engineered or wild type strain. Details on the methods and results are below.

Exemplary Realization—gDNA

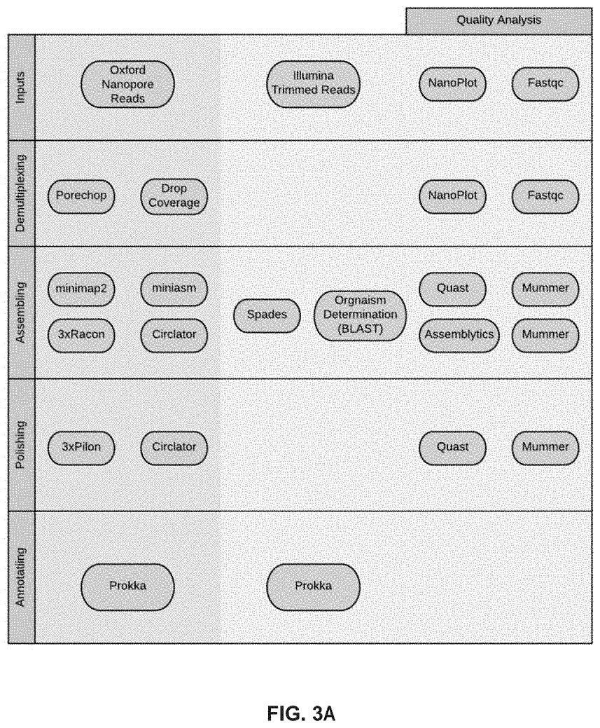

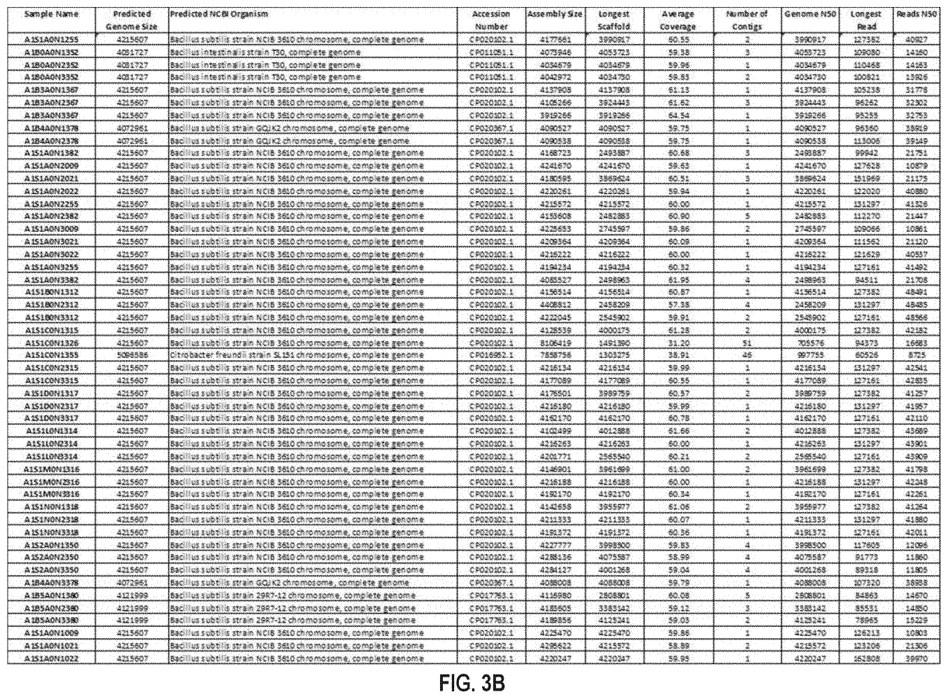

Datasets (e.g., Illumia reads and oxford reads) for 50 samples passed QC standards, and data is provided in a summary table in FIG. 3B. It was determined that the number of reads above Q30 was 87±0.3% for the 50 samples where the lowest quality score observed was 81% of reads for sample A1B4A0N1378 which is still higher than the minimum requirement of 80% of reads must be greater than Q30. FIG. 3A shows a list of software used for QC and genome assembly.

In the exemplary realization, assemblies were generated for all samples using both read types. Two types of genome assemblies were provided for PanOCT analysis. One type of assembly was generated with only Illumina reads using Spades since these assemblies are similar to the datasets the classifier was trained on. Additionally, 50 assemblies were generated with Oxford long-reads and polished with Illumina reads were generated. Averaging all 50 samples, the average number of contigs from ONT polished assemblies was 4±9 contigs and genome N50's of 3.7±0.8 megabases. On average using only Illumina reads for assemblies, samples assembled to 47±34 contigs and genome N50's of 0.82±0.3 megabases. On average genome assemblies took 40.78±14 wall clock minutes to assemble and 161±37 CPU minutes to assemble.

Furthermore, the de novo assembly pipeline also determines the closest matching genome in the NCBI database (FIG. 3B). The NCBI fasta genome is downloaded and used as a reference genome for whole genome comparisons to the sample assembly. After genome alignment, the genomic insertions and deletions within the sample genome are parsed, 6-frame translated and blastp is performed. Finally, the blast hits are used to determine the function of the insertion. At this point the blast hits can be visually inspected by a researcher. Additionally or alternatively, automated word cloud generation can be provided.

### Exemplary Realization—BioVelocity

In the exemplary realization, Illumina and MinION reads for 50 paired-end sample were received. Each sample included R1 and R2 for the two ends of the pair. Each end was evaluated independently, but preference was given to R1 over R2 due to increased errors on R2 (need Ref). Each read was processed through the BioV platform by aligning against three indexes: Bacillus sub. sub., all NCBI bacteria, and BioBricks (described below).

The first index is a collection of all published BioBrick sequences, excluding those labeled as naturally occurring in Bacillus subtilis. This analysis produced a list of all BioBricks that aligned to a read with at least 95% accuracy. A count of these alignments is then merged with the output of the NLP (BioBricks column in both images).

The second index is a collection of 146 Bacillus subtilis subtilis references sequences. The BioV comparison produced a count of reads that perfectly aligned, partially aligned (95-99%), and did not align to the index. The references that partially aligned are reported with alignment scores and SNPs are reported. A summary statics analysis was run that calculates the ratio between partially aligned reads and all reads, which is fed into the classifier (described later in this report).

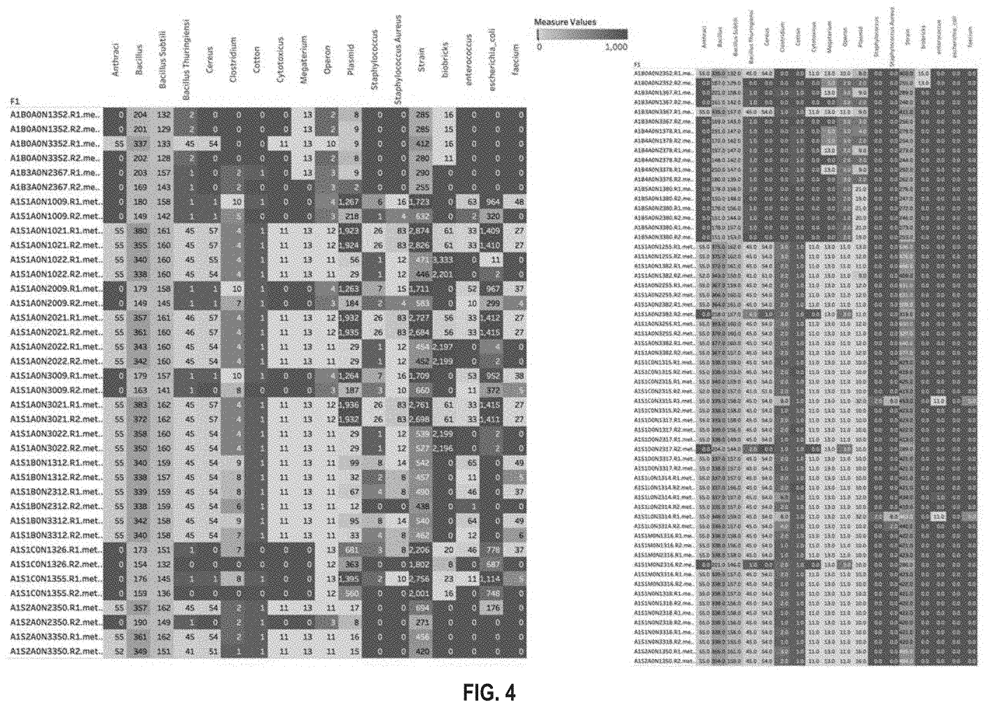

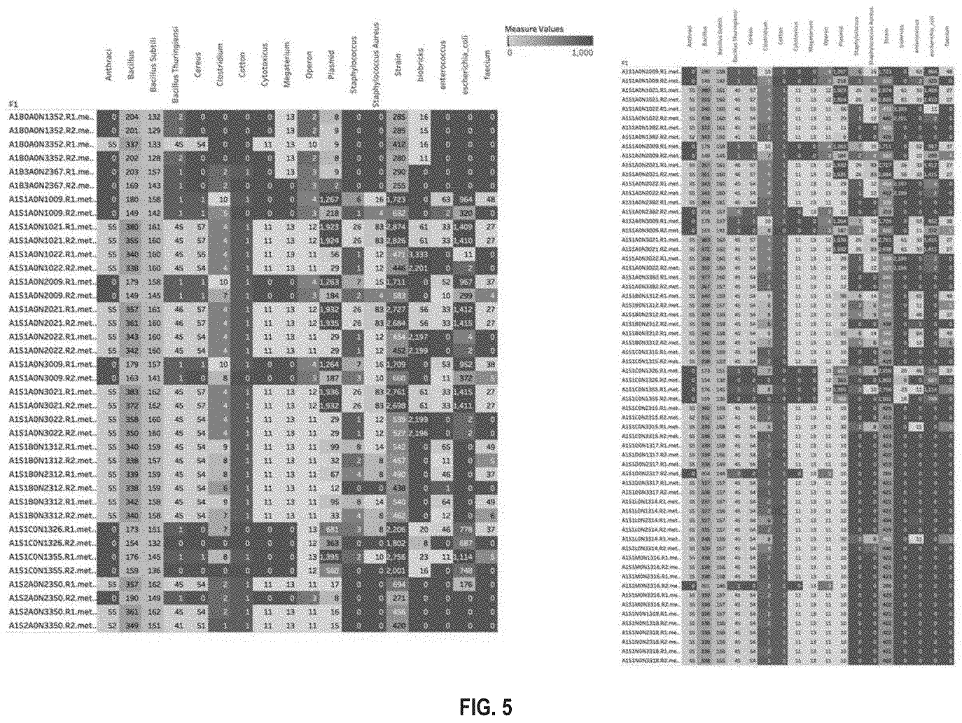

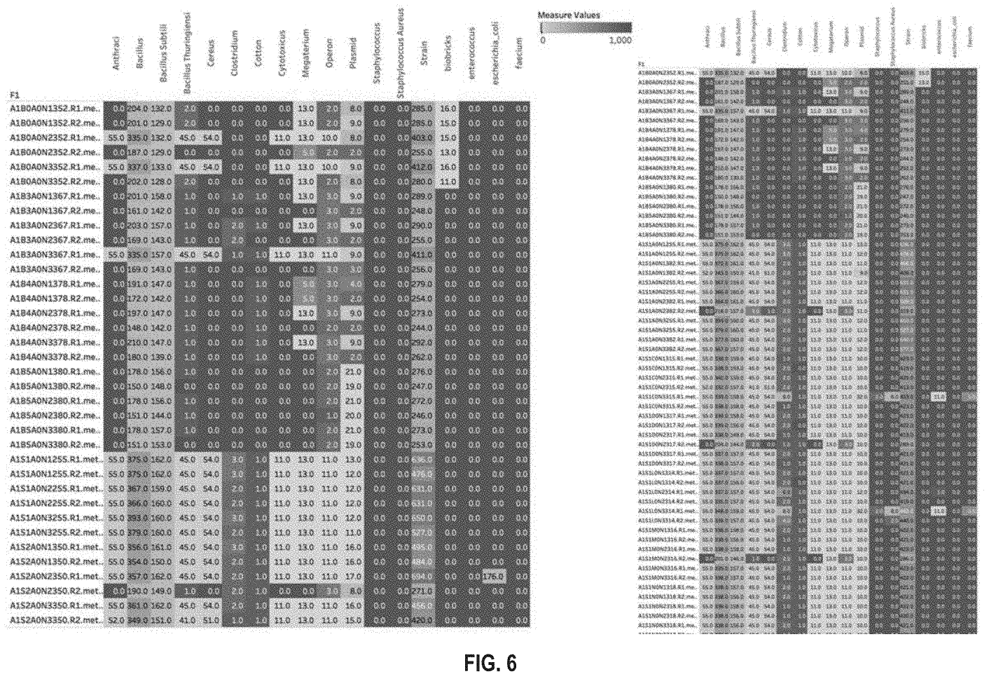

The third index included a large collection of NCBI's bacterial sequences using the GenBank collection of complete chromosomes and plasmids. Analysis with BioV produced a metagenomics output listing each reference that aligned to a read with at least 95% accuracy. The reported aligned references were processed through a natural language processing (NLP) library in R named TM (BioV+NLP), available at https://cranr.-project.org/web/packages/tm/tm.pdf. One function of this library counts the occurrence of key terms for each sample as defined by the user. Those are then mapped using a heatmap graphic to represent the count of each key term across all samples. The NLP strips out numbers and stems each work to remove modifiers to improve matching of terms with the same meeting using different tense or other modifiers. The heatmap graphic is produced using Tableau for ease of filtering and analysis. Critical key terms had been previously identified during our research under the FELIX program and include: ‘plasmid’, ‘Staphylococcus aureus’, ‘enteroccoccus’, ‘Escherichia coli’, ‘faecium’. Low occurrences of these terms correlate with wild-type and high occurrences correlate with engineering (FIGS. 4, 5, and 6).

Codon optimization was performed on reads which poorly aligned (<95%) to the B. sub. subtilis reference index. Reads were blasted against an all B. sub. Proteins database and an all bacterial proteins database (Uniprot). All hits with 100% alignment were taken, and the subject names (-outfmt sallnames) were processed via the NLP engine and also fed into the classifier.

### Exemplary Realization—BioVelocity Results

In some embodiments, the initial output of the BioV system that was not used in the classifier was limited to the metagenomics output with the NLP post processing. FIG. 4 shows the predictions for engineered vs wild-type using only the complete analysis, which included the classifier combined with BioV+NLP.

Exemplary Realization—PanOCT (FELIX Bacillus subtilis Pan-Genome Graph (PGG) Analysis)

The design and implementation of a PGG based analysis of synthetically engineered genomes required multiple stages and inputs. For the first prototype/model species Bacillus subtilis was chosen. The first step was to determine and retrieve all Bacillus subtilis genomes from RefSeq. The second step was to run the JCVI pan-genome pipeline to generate a PGG for Bacillus subtilis using the RefSeq genomes and annotations. The third step was to develop the programs necessary to consistently annotate genomes using a PGG. These programs were then combined into a wrapper script along with other helper programs to iteratively regenerate a PGG based on the PGG annotated genomes until a stable PGG was obtained based on consistent PGG annotation. The stable PGG could then be used to annotate novel genomes and detect which regions of the genome (both genes and edges between genes) were either completely novel or novel alleles. A feature vector was defined to pass relevant information to a classifier for training to recognize synthetic engineering versus natural occurrence. More voluminous data, such as multiple sequence alignments, was also generated for more detailed follow on analysis. For simulated genomes some of this data could be used to determine sensitivity and specificity of the PGG annotation to recognize novel genes/edges and novel alleles of both.

Determining the RefSeq Bacillus subtilis genomes for the initial PGG.

In the exemplary realization, 169 genomes labeled B. subtilis from RefSeq at NCBI were downloaded. These genomes were used as input for the JCVI pan-genome pipeline which generates a PGG among other files. Additional descriptions can be found in Inman, J. M., et al., Large-Scale Comparative Analysis of Microbial Pan-genomes using PanOCT. Bioinformatics, 2018; Fouts, D. E., et al., PanOCT: automated clustering of orthologs using conserved gene neighborhood for pan-genomic analysis of bacterial strains and closely related species. Nucleic Acids Res, 2012. 40(22): p. e172; and Chan, A. P., et al., A novel method of consensus pan-chromosome assembly and large-scale comparative analysis reveal the highly flexible pan-genome of Acinetobacter baumannii. Genome Biol, 2015. 16: p. 143. Based on the literature and the average nucleotide identity (ANI) based tree generated by the pan-genome pipeline, there were 8 B. subtilis subsp. inaquosorum genomes, 13 B. subtilis subsp. spizizenii genomes, 5 B. subtilis subsp. stecoris genomes, 130 B. subtilis subsp. subtilis genomes, 2 B. subtilis subsp. unkown1 genomes, 1 B. subtilis subsp. unkown2 genome, 8 Bacillus velezensis genomes, and 2 Bacillus atrophaeus genomes. Using typical ANI thresholds for subspecies (97-98%) and species (95-96%), B. subtilis subsp. inaquosorum, B. subtilis subsp. spizizenii, B. subtilis subsp. unkown1, and B. subtilis subsp. unkown2 would be separate species since they drop below 95% ANI to each other and B. subtilis subsp. spizizenii would need to be split into 3 subspecies. In addition to the non-subtilis species to be removed the pan-genome run also helped reveal that the same strain (the type strain) was sequenced multiple times resulting in 8 largely duplicated duplicate genomes.

Further, 167 Bacillus type strains were pulled from Refseq to be used for a Bacillus pan-genome and for “typing” all Bacillus genomes in Refseq using MASH. Additional descriptions can be found at Sutton, G. G., et al., Enterobacter hormaechei subsp. hoffmannii subsp. nov., Enterobacter hormaechei subsp. xiangfangensis comb. nov., Enterobacter roggenkampii sp. nov., and Enterobacter muelleri is a later heterotypic synonym of Enterobacter asburiae based on computational analysis of sequenced Enterobacter genomes. F1000Res, 2018. 7: p. 521; Ondov, B. D., et al., Mash: fast genome and metagenome distance estimation using MinHash. Genome Biol, 2016. 17(1): p. 132; and Rooney, A. P., et al., Phylogeny and molecular taxonomy of the Bacillus subtilis species complex and description of Bacillus subtilis subsp. inaquosorum subsp. nov. Int J Syst Evol Microbiol, 2009. 59(Pt 10): p. 2429-36. This helped determine which genomes are B. subtilis, which are closely related, which are distant Bacillus, and which are not Bacillus. The following papers indicated Bacillus species which were proposed to be in the B. subtilis complex: Fan, B., et al., Bacillus amyloliquefaciens, Bacillus velezensis, and Bacillus siamensis Form an “Operational Group B. amyloliquefaciens” within the B. subtilis Species Complex. Front Microbiol, 2017. 8: p. 22; and Alina, S. O., Constantinscu, Florica, and Petruta, Cornea Calina, Biodiversity of Bacillus subtilis group and beneficial traits of Bacillus species useful in plant protection. Romanian Biotechnological Letters, 2015. 20(5): p. 10737-10750. To include the expected B. subtilis complex species a cutoff of 73% ANI was used with all other genomes compared to B. subtilis subsp. subtilis falling at or below 72% ANI. There was no clean cutoff for all of the genomes in the B. subtilis complex but the highest ANI outside of the complex to the complex is 72.35% ANI and the lowest within the complex is 72.06% ANI.

Six clades are observed:

| Species and strain | ANI |
|---|---|
| B. subtilis: B. subtilis subsp subtilis, B. | >88% ANI to B. subtilis |
| subtilis subsp. stecoris, B. subtilis subsp. | subsp. subtilis |
| inaquosorum, B. subtilis subsp. spizizenii, | |
| B. tequilensis, B. vallismortis, B. mojavensis, | |
| B. halotolerans | |
| B. atrophaeus | >80% ANI to B. subtilis |
| | subsp. subtilis |
| B. amyloliquefaciens: B. amyloliquefaciens, | >78% ANI to B. subtilis |
| B. siamensis, B. velezensis, B. nakamurai | subsp. subtilis |
| B. licheniformis: B. lichenif>ormis, B. | >74% ANI to B. subtilis |
| haynesii, B. paralicheniformis, B. swezeyi, | subsp. subtilis |
| B. glycinifermentans, B. sonorensis | |
| B. pumilus: B. pumilus, B. cellulasensis, | >73% ANI to B. subtilis |
| B. altitudinis, B. xiamenensis, B. | subsp. subtilis |
| australimaris, B. safensis | |
| B. gobiensis | 73.06% ANI to B. subtilis |
| | subsp. subtilis |

More species may be represented in these clades, if the type strains are available.

Over 3000 Bacillus genomes were pulled from RefSeq and compared against all 167 Bacillus type strains using MASH a fast ANI approximation. All genomes which had their best matches to type strains within the six clades of the B. subtilis complex were assigned to those clades and used for a hierarchical pan-genome run of 695 genomes in 6 batches.

For the B. subtilis batch (207 genomes), 146 genomes are B. subtilis subsp. subtilis (blue), 11 genomes are B. subtilis subsp. stecoris (green), 14 genomes are B. subtilis subsp. spizizenii (purple), 9 genomes are B. subtilis subsp. inaquosorum (red), 18 genomes are B. halotolerans (teal), and there are 5 other clades of size 1-3 of various assignment.

For the B. atrophaeus batch all 31 genomes formed a tight clade.

For the B. amyloliquefaciens batch (211 genomes), 13 genomes are B. amyloliquefaciens (green) although 56 were submitted by that name in this batch, 187 genomes are B. velezensis (blue), 9 genomes are B. siamensis, and 2 genomes are B. nakamurai. All four species are tightly clustered with ANI values >97.5% within each species. B. amyloliquefaciens, B. velezensis, and B. siamensis are all approximately 94% ANI to each other. B. nakamurai are approximately 86% ANI to the other three species.

For the B. licheniformis batch (107 genomes), 69 genomes are B. licheniformis (blue), 1 genome is B. haynesii which is just outside of the B. licheniformis clade, 24 genomes are B. paralicheniformis (red), 5 genomes are B. sonorensis (purple), 5 genomes are B. glycinifernentans (green), two genomes are B. swezeyi (teal), and 1 genome is unidentified. Names were relatively consistent for this batch.

For the B. pumilus batch (138 genomes), 52 genomes form a fairly tight clade but there are two subclades, one subclade we are designating B. cellulasensis has 35 genomes (green) and contains the type strain for B. cellulasensis but also contains a type strain for B. altitudinis, the other subclade we are designating B. altitudinis has 17 genomes (purple) and contains another type strain for B. altitudinis, 39 genomes are B. pumilus (blue), 40 genomes are B. safensis (red), 2 genomes are B. xiamenensis (teal), 3 genomes are B. zhangzhouensis (orange), 1 genome is B. australimaris, and 1 genome is unidentified.

For initial testing only B. subtilis subsp. subtilis genomes were used. A pan-genome with the 146 genomes available in RefSeq was constructed. There are 12668 gene clusters of which 3593 are core clusters using a threshold of 95% for core, 4372 are accessory, and 4703 are unique to a single genome. There are 29798 edges in the Pan-Genome Graph (PGG). These 146 genomes were reannotated based on the initial PGG from which a new PGG was constructed and this process iterated until the PGG stabilized. The original PGG stats were: 4703 size 1 clusters, 7965 shared (size >1) clusters, 625732 genes in the shared clusters, 3593 core (95% of genomes) clusters, 5402 size 1 edges, 9497 shared edges, 619231 edge instances in the shared edges, and 3271 core edges. The refined PGG stats after stabilization were: 2147 size 1 clusters, 7986 shared (size >1) clusters, 651301 genes in the shared clusters, 3776 core clusters, 3649 size 1 edges, 9322 shared edges, 645457 edge instances in the shared edges, and 3526 core edges. Both the cluster and edge stats can be improved as follows: the number of size 1 clusters/edges significantly decreased due to some bogus gene calls being eliminated and some becoming shared with other genomes; the number of core clusters/edges significantly increased showing an improvement in the consistency of annotation across all genomes; and the number of genes/edges in clusters/edge instances greatly increased again indicating a much more consistent annotation.

The completion of the stable PGG based on reannotation of the 146 genomes in the PGG allowed the generation of a large batch of consistently annotated genomes and an associated feature vector to be used by the classifier. Three separate batches were run: the 146 genomes in the PGG but subtracting the genome to be annotated from the PGG for generating the feature vector; the 20 engineered or wild type genomes generated and sequenced at JCVI for three different assembly types each (Illumina only, MinION only, and MinION polished with Illumina); and 880 synthetic genomes with modifications from the medoid genome of the 146 in the PGG.

The classifier feature vector output from the pan-genome tools consisted of the following features:

TABLE 1
Classifier feature vector output
Description

the number of unique clusters as annotated by the PGG
the number of genes with unique alleles in clusters with
penetrance 0-25%
the number of genes with unique alleles in clusters with
penetrance 25-75%
the number of genes with unique alleles in clusters with
penetrance 75-100%
the number of unique edges
the number of edges with unique alleles in clusters with
penetrance 0-25%
the number of edges with unique alleles in clusters with
penetrance 25-75%
the number of edges with unique alleles in clusters with
penetrance 75-100%
the number of genes that are shorter than expected
the number of genes that are longer than expected
the number of edges that are shorter than expected
the number of edges that are longer than expected
the number of protein coding genes which appear to be
frameshifted
clusters with penetrance 75% or higher which are missing (not
including core)
edges with penetrance 75% or higher which are missing (not
including core)
singleton core clusters which are missing
core edges which are missing
number of (nonsingleton) shared clusters found includes core
number of (nonsingleton) shared edges found includes core
number of genes whose ANI is less than 90% to the cluster
medoid
number of rearrangements (novel core edges)
ANI measured against all of the cluster medoids

In some embodiments, the first full run through of the PGG code base illustrated that novel genes/edges are still being called for the 146 genomes that make up the PGG even after using the stabilized PGG. This can be due to under annotation of the original RefSeq annotation. The pipeline could ignore these genes and leave them as novel edge alleles but for new genomes this might be a less enticing option since inserted genes or gene cassettes would then end up as part of edges (those edges would be flagged as unique alleles and too long, but this would be suboptimal). This issue can be addressed as follows: allowing the novel genes to be included in edges, including the novel genes as part of the PGG stabilization so that they would not be novel genes after stabilization, including either or both of a gene caller or raw orf caller as input to help delineate gene boundaries in addition to the homology being used, and as considered before allowing multiple exemplars for diverse gene clusters rather than just the medoid.

The fifty genomes were assembled in two ways: just Illumina reads and ONT reads followed by polishing with Illumina. Both assembly types for a total of 100 samples were run through the pan-genome tools to generate feature vectors for the classifier.

The testing indicates that more than just the feature vector for the classifier is desirable for elucidating specific engineered changes and possibly intent. The pangenome tools generate much more data than just the feature vector which can be reviewed manually for rich information. The objective is to effectively and efficiently process this to flag only the most relevant data for efficient review.

### Classifier

The generation of classifiers to distinguish natural sequences from engineered sequences requires sufficient examples for model training. One can use actual genomes generated using synthetic biology vs. known wild-type genomes. However, actual synthetic biology requires considerable time, effort, and expense. Therefore, in our effort we focused on the development of in silico methods to generate simulated synthetic biology examples. This approach requires 1) knowledge of typical methods of engineering, 2) a faithful simulation of illumina read error, 3) and a reference genome for the purpose of making errors.

In an exemplary realization, a PGG was generated to determine a Bacillus subtilis reference genome (3610a) as well as a list of core regions not to be changed (as doing so would likely affect viability). This genome became the basis of the simulated changes. Twenty different types of in silico synthetic changes were generated. Each of these types of changes was applied to the reference genome 1 to N times, typically with 20 replicates each. The types of engineering are shown in Table 2. These are described as follows. The “gene deletion” operator, identifies a non-core gene at random and deletes the gene from the genome. The “gene duplication” operator, identifies a non-core gene at random and inserts that back elsewhere at a random location into the genome.

The “Insert GFP” operator inserts a green fluorescent protein gene used previously for synthetic biology by JCVI at a random location in the genome. The “Insert GFP with CRISPR/cas9” does the same but using a cas9 operator. “Operon deletion” identifies an operon a random and deletes the entire operon from the genome simulating a larger scale deletion. “Promoter deletion” focuses on the region −1 to −70 upstream of an ORF chosen at random and deletes this from the genome. We generated three types of single nucleotide changes including SNPs and indels randomly distributed. The “Tn5-Puro” insertion inserts a cassette including Tn5 and puromycin at a random location in the genome (avoiding core regions). Simulating other types of edits, the “delete first half of gene” and “delete last half of gene” operators identify a gene at random, then delete a random percentage between 10-50% of either the first or last half respectively. The “delete random length of promoter (50-80 nt)” has a similar function by choosing a promoter at random and deleting a random length between 50-80 nt in that promoter. Additional insertions/deletions/SNPs were generated including a random choice of insertion/deletion in a randomly chosen promoter or ORF. Over the course of the effort the simulations became more specific to types of effort and intent, including insertion of SNPs in ORFs specifically to change amino acids to alanine, or through the use of homoendonucleases such as I-Sce to delete sections of a genome, or through the replacement of a gene with a GFP BioBrick. The 20^{th} type of change modeled the more complex MoClo system (addgene) allowing for the insertion sets of promoters, genes, etc. as a system. Each of these simulations was generated and then evaluated by NSI and JCVI before being applied 1 or more times per genome with multiple replicates to generate a diverse yet realistic set of examples for training spanning small scale changes (SNPs, indels) to larger scale changes with the expectation that larger changes would be easier to detect, and with the expectation that multiple changes per genome would also be easier to detect.

For each of these changes, once generated we used a function such as dwgsim, available at https://github.com/nh13/DWGSIM, to chop the edited reference genome into fragments representative of illumina reads. At first these were simulated using a default error profile in dwgsim. However, comparison of this error to actual illumina reads indicated that both the profile and the amount of error in the simulated reads was insufficient. Iterated adjustment and review led to a more reasonable error rate, and a switch to a “V” shaped error profile available in dwgsim with increased error at the 5′ and 3′ ends of each read. This revised error rate was then used to regenerate all examples to date and used to generate the remaining new types of simulated changes shown in Table 2. The data was used directly by PanOCT for feature generation, while the data was then assembled for use by BioV. Features generated by these two approaches were then captured for all synthetic sequences as well as natural sequences and provided back to NSI for classifier development (see below).

### Classifier Development and Evaluation

The features generated by PanOCT and BioV for both synthetic (engineered) and natural sequences were initially evaluated to determine their ability on a feature-by-feature basis to separate these two categories. Given the large number of examples of synthetic sequences (based on one reference genome 3610a) vs. 146 natural genomes from a wide diversity of Bacillus, this separation was difficult to achieve. Training of neural networks as classifiers utilized these features as input to classify sequences as either synthetic or engineered. NSI evaluated a variety of sample balancing approaches to ensure that the difference in samples between synthetic and engineered had minimal effect on classifier performance. The data were divided repeatedly into training, testing, and validation sets. Evolutionary computation was used to optimize the weights associated with a population of neural networks (a process known as evolved neural networks) to reduce error on the output decision. This process also affords the opportunity to do feature selection simultaneously with model optimization. As a result, we identified that of the 21 possible PanOCT features, models that used 10 features performed the best. Similar evaluation for BioV demonstrated that the R1 Illumina inputs performed better than the R2 Illumina inputs. The best classifiers from this process of feature selection and model optimization were used for an internal end-to-end evaluation. During the end-to-end and T&E1 process it became clear that 1) the diversity of our training examples was low reduced and that additional reference genomes would improve classifier performance, and 2) that PanOCT and BioV were identifying changes of different types, which could be very valuable as input to a single integrated classifier.

During the exemplary realization, it became clear that a decision tree type approach can be advantageous, if BioV (or other) algorithm (or human expert) can easily identify the presence/absence of clear indicators of synthetic change such as the insertion of non-prokaryotic DNA in a prokaryotic genome, etc. These key flags could be used to call a sample as synthetic quickly, leaving more difficult decisions to the classifier.

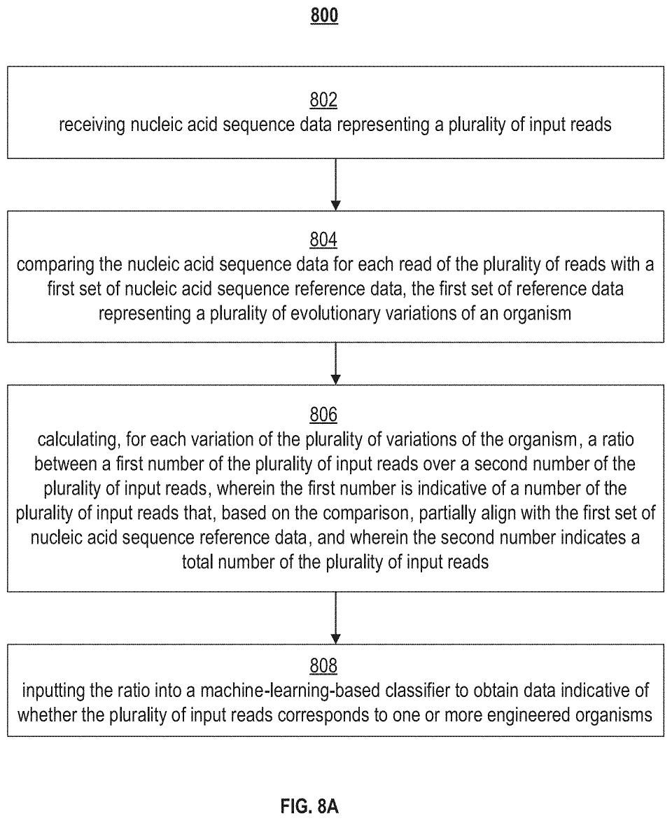

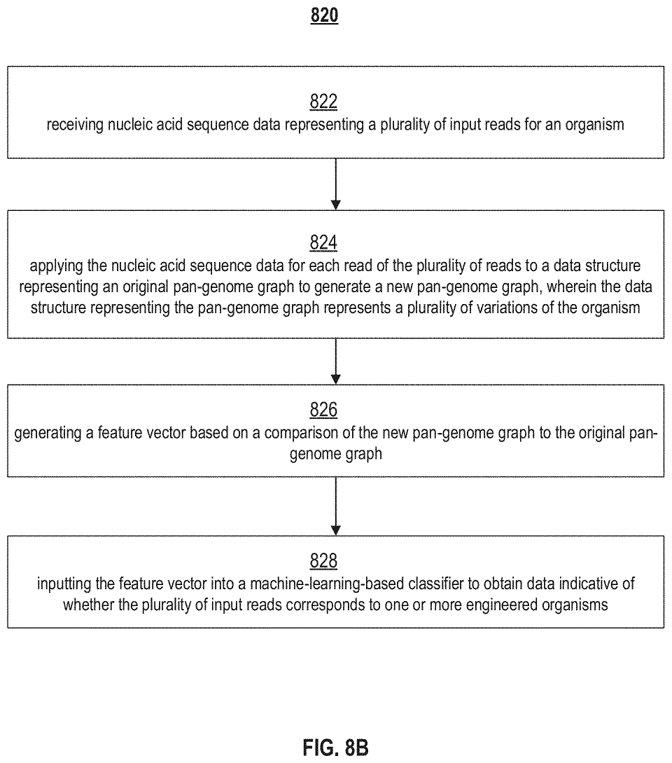

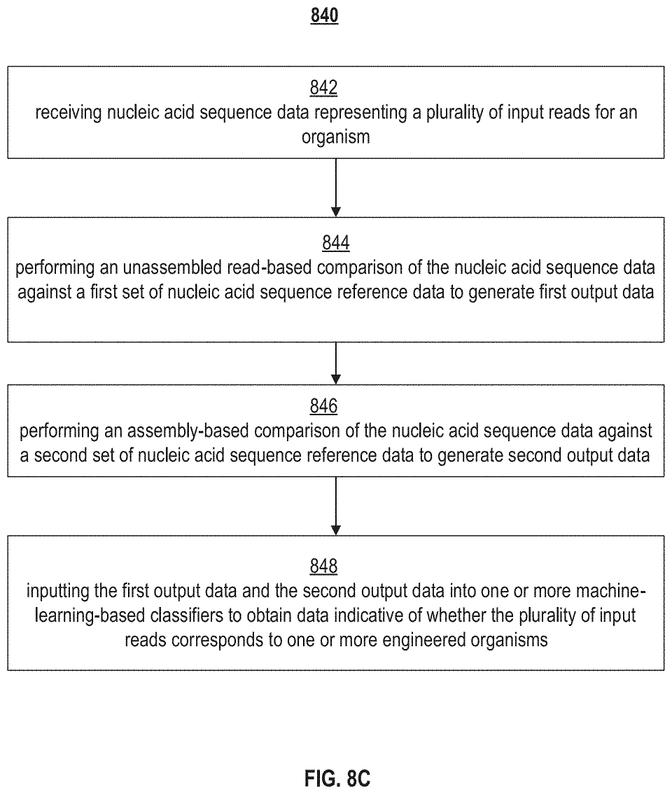

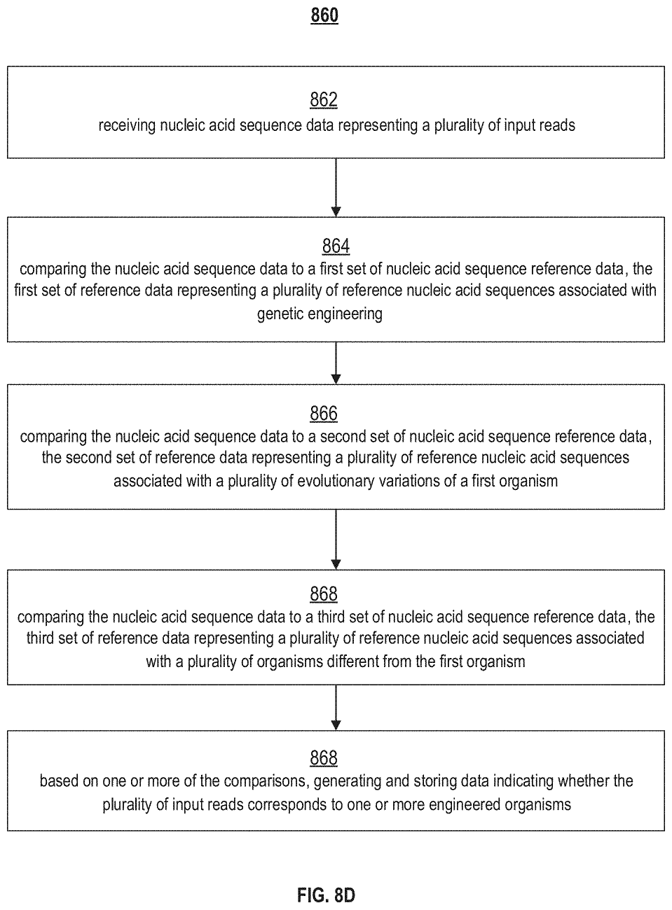

FIGS. 8A-D illustrates processes 800, 820, 840, and 860, according to various examples. Each of the processes can be performed, for example, using one or more electronic devices implementing a software platform. In some examples, each process can be performed using a client-server system, and the blocks of the process can be divided up in any manner between the server and a client device. In other examples, the blocks of each process can be divided up between the server and multiple client devices. Thus, while portions of each process are described herein as being performed by particular devices of a client-server system, it will be appreciated that the processes are not so limited. In other examples, each process can be performed using only a client device (e.g., user device 900) or only multiple client devices. In each process, some blocks are, optionally, combined, the order of some blocks is, optionally, changed, and some blocks are, optionally, omitted. In some examples, additional steps may be performed in combination with each process. Accordingly, the operations as illustrated (and described in greater detail below) are exemplary by nature and, as such, should not be viewed as limiting.

In the exemplary process 800, at block 802, the system receives nucleic acid sequence data representing a plurality of input reads. At block 804, the system compares the nucleic acid sequence data for each read of the plurality of reads with a first set of nucleic acid sequence reference data, the first set of reference data representing a plurality of evolutionary variations of an organism. At block 806, the system calculates, for each evolutionary variation of the plurality of variations of the organism, a ratio between a first number of the plurality of input reads over a second number of the plurality of input reads, wherein the first number is indicative of a number of the plurality of input reads that, based on the comparison, align with the first set of nucleic acid sequence reference data within a predefined range, and wherein the second number indicates a total number of the plurality of input reads. At block 808, the system inputs the ratio into a machine-learning-based classifier to obtain data indicative of whether the plurality of input reads corresponds to one or more engineered organisms.

In the exemplary process 820, at block 822, the system receives nucleic acid sequence data representing a plurality of input reads for an organism. At block 824, the system applies the nucleic acid sequence data for each read of the plurality of reads to a data structure representing an original pan-genome graph to generate a new pan-genome graph, wherein the data structure representing the pan-genome graph represents a plurality of variations of the organism. At block 826, the system generates a feature vector based on a comparison of the new pan-genome graph to the original pan-genome graph. At block 828, the system inputs the feature vector into a machine-learning-based classifier to obtain data indicative of whether the plurality of input reads corresponds to one or more engineered organisms.

In the exemplary process 840, at block 842, the system receives nucleic acid sequence data representing a plurality of input reads for an organism. At block 844, the system performs an unassembled read-based comparison of the nucleic acid sequence data against a first set of nucleic acid sequence reference data to generate first output data. At block 846, the system performs an assembly-based comparison of the nucleic acid sequence data against a second set of nucleic acid sequence reference data to generate second output data. At block 848, the system inputs the first output data and the second output data into one or more machine-learning-based classifiers to obtain data indicative of whether the plurality of input reads corresponds to one or more engineered organisms.

In the exemplary process 860, at block 862, the system receives nucleic acid sequence data representing a plurality of input reads. At block 864, the system compares the nucleic acid sequence data to a first set of nucleic acid sequence reference data, the first set of reference data representing a plurality of reference nucleic acid sequences associated with genetic engineering. At block 866, the system compares the nucleic acid sequence data to a second set of nucleic acid sequence reference data, the second set of reference data representing a plurality of reference nucleic acid sequences associated with a plurality of evolutionary variations of a first organism. At block 868, the system compares the nucleic acid sequence data to a third set of nucleic acid sequence reference data, the third set of reference data representing a plurality of reference nucleic acid sequences associated with a plurality of organisms different from the first organism. At block 868, based on one or more of the comparisons, the system generates and stores data indicating whether the plurality of input reads corresponds to one or more engineered organisms.

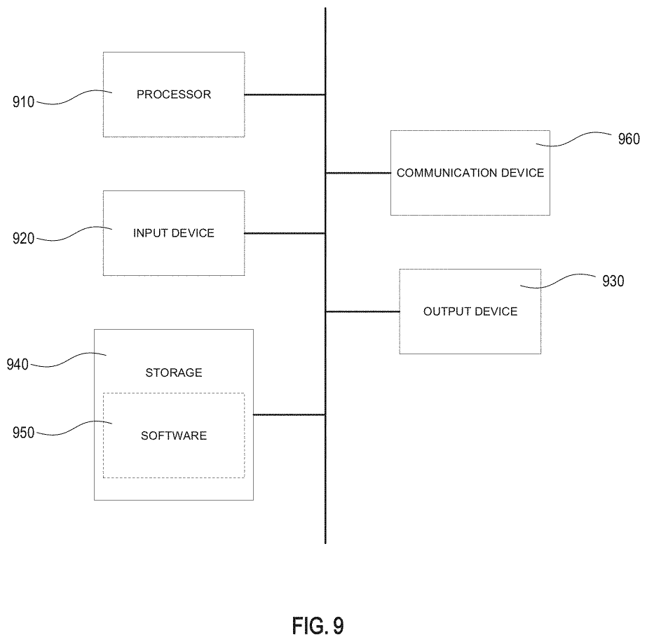

The operations described above with reference to FIGS. 8A-D are optionally implemented by components depicted in FIG. 9. It would be clear to a person having ordinary skill in the art how other processes are implemented based on the components depicted in FIG. 9.

FIG. 9 illustrates an example of a computing device in accordance with one embodiment. Device 900 can be a host computer connected to a network. Device 900 can be a client computer or a server. As shown in FIG. 9, device 900 can be any suitable type of microprocessor-based device, such as a personal computer, workstation, server or handheld computing device (portable electronic device) such as a phone or tablet. The device can include, for example, one or more of processor 910, input device 920, output device 930, storage 940, and communication device 960. Input device 920 and output device 930 can generally correspond to those described above, and can either be connectable or integrated with the computer.

Input device 920 can be any suitable device that provides input, such as a touch screen, keyboard or keypad, mouse, or voice-recognition device. Output device 930 can be any suitable device that provides output, such as a touch screen, haptics device, or speaker.

Storage 940 can be any suitable device that provides storage, such as an electrical, magnetic or optical memory including a RAM, cache, hard drive, or removable storage disk. Communication device 960 can include any suitable device capable of transmitting and receiving signals over a network, such as a network interface chip or device. The components of the computer can be connected in any suitable manner, such as via a physical bus or wirelessly.

Software 950, which can be stored in storage 940 and executed by processor 910, can include, for example, the programming that embodies the functionality of the present disclosure (e.g., as embodied in the devices as described above).

Software 950 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 940, that can contain or store programming for use by or in connection with an instruction execution system, apparatus, or device.

Software 950 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

Device 900 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 900 can implement any operating system suitable for operating on the network. Software 950 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

What is claimed is:

1. A computer-enabled method for determining whether an organism is genetically engineered, the method comprising:
receiving nucleic acid sequence data representing a plurality of input reads;
comparing the nucleic acid sequence data for each read of the plurality of reads with a first set of nucleic acid sequence reference data, the first set of reference data representing a plurality of evolutionary variations of an organism;
calculating, for each evolutionary variation of the plurality of variations of the organism, a ratio between a first number of the plurality of input reads over a second number of the plurality of input reads,
wherein the first number is indicative of a number of the plurality of input reads that, based on the comparison, align with the first set of nucleic acid sequence reference data within a predefined range, and wherein the second number indicates a total number of the plurality of input reads;
inputting the ratio into a machine-learning-based classifier to obtain data indicative of whether the plurality of input reads corresponds to one or more engineered organisms.

2. The method of claim 1, further comprises obtaining the first number, wherein obtaining the first number comprises:
comparing an input read of the plurality of input reads with nucleic acid sequence reference data corresponding to a respective evolutionary variation of the plurality of variations to obtain a degree of matching between the input read and the nucleic acid sequence reference data corresponding to the respective evolutionary variation;
determining whether the degree of matching is within the predefined range;
in accordance with a determination that the degree of matching is within the predefined range, incrementing the first number; and
in accordance with a determination that the degree of matching is not within the predefined range, foregoing incrementing the first number.

3. The method of claim 1, wherein the predefined range comprises an upper threshold, and wherein the upper threshold is equal to or higher than 99%.

4. The method of claim 1, wherein the predefined range comprises a lower threshold, and wherein the lower threshold is equal to or lower than 95%.

5. The method of claim 1, wherein the data indicative of whether the plurality of input reads corresponds to one or more engineered organisms comprises one or more confidence scores.

6. A computer-enabled method for determining whether an organism is genetically engineered, the method comprising:
receiving nucleic acid sequence data representing a plurality of input reads for an organism;
performing an unassembled read-based comparison of the nucleic acid sequence data against a first set of nucleic acid sequence reference data to generate first output data;
performing an assembly-based comparison of the nucleic acid sequence data against a second set of nucleic acid sequence reference data to generate second output data;
inputting the first output data and the second output data into one or more machine-learning-based classifiers to obtain data indicative of whether the plurality of input reads corresponds to one or more engineered organisms.

7. The method of claim 6, wherein the first set of nucleic acid sequence reference data represents a plurality of evolutionary variations of the organism.

8. The method of claim 7, wherein performing an unassembled read-based comparison of the nucleic acid sequence data against a first set of nucleic acid sequence reference data to generate first output data comprises:
calculating, for each evolutionary variation of the plurality of variations of the organism, a ratio between a first number of the plurality of input reads over a second number of the plurality of input reads,
wherein the first number is indicative of a number of the plurality of input reads that, based on the comparison, align with the first set of nucleic acid sequence reference data within a predefined range, and wherein the second number indicates a total number of the plurality of input reads.

9. The method of claim 8, further comprises:
determining a degree of matching between the nucleic acid sequence data and the first set of nucleic acid sequence reference data;
in accordance with a determination that the degree of matching is below a threshold, comparing the nucleic acid sequence data to the second set of nucleic acid sequence reference data, and
in accordance with a determination that the degree of matching is not below a threshold, foregoing comparing the nucleic acid sequence data to the second set of nucleic acid sequence reference data.

10. A computer-enabled method for determining whether an organism is genetically engineered, the method comprising:
receiving nucleic acid sequence data representing a plurality of input reads;
comparing the nucleic acid sequence data to a first set of nucleic acid sequence reference data, the first set of reference data representing a plurality of reference nucleic acid sequences associated with genetic engineering;
comparing the nucleic acid sequence data to a second set of nucleic acid sequence reference data, the second set of reference data representing a plurality of reference nucleic acid sequences associated with a plurality of evolutionary variations of a first organism;
comparing the nucleic acid sequence data to a third set of nucleic acid sequence reference data, the third set of reference data representing a plurality of reference nucleic acid sequences associated with a plurality of organisms different from the first organism;
based on one or more of the comparisons, generating and storing data indicating whether the plurality of input reads corresponds to one or more engineered organisms.
